(19) United States
(12) Patent Application Publication
(10) Pub. No.: US 2015/0211066 A1
(43) Pub. Date: Jul. 30, 2015

(54) Methods and Compositions for Correlating Genetic Markers with Sudden Cardiac Death Risk

(71) Applicant: Duke University, Durham, NC (US)

(72) Inventors: Geoffrey S. Pitt, Durham, NC (US); Albert Y. Sun, Durham, NC (US)

(21) Appl. No.: 14/534,703

(22) Filed: Nov. 6, 2014

Related U.S. Application Data
(63) Continuation of application No. 13/315,855, filed on Dec. 9, 2011, now abandoned.
(60) Provisional application No. 61/421,654, filed on Dec. 10, 2010.

(51) Int. Cl. C12Q 1/68

(52) U.S. Cl. CPC C12Q 1/6883; C12Q 2600/156; C12Q 2600/112

(57) ABSTRACT

The present invention provides a method of identifying a subject as having an increased risk of sudden cardiac death or cardiac arrhythmia by detecting in the subject the presence of various polymorphisms associated with an increased risk of sudden cardiac death or cardiac arrhythmia.

### STATEMENT OF PRIORITY

This application is a continuation application of, and claims priority to, U.S. application Ser. No. 13/315,855 filed Dec. 9, 2011 (abandoned), which claims the benefit, under 35 U.S.C. §119(e), of U.S. Provisional Application Ser. No. 61/421,654, filed Dec. 10, 2010, the entire contents of each of which are incorporated by reference herein.

### STATEMENT OF GOVERNMENT SUPPORT

This invention was made with government support under Grant Nos. T32HL007101-35, R01 HL71165 and R01 HL088089 awarded by the National Institutes of Health. The government has certain rights in the invention.

### FIELD OF THE INVENTION

The present invention provides methods and compositions directed to identification of genetic markers associated with sudden cardiac death.

### BACKGROUND OF THE INVENTION

African Americans disproportionately suffer from heart failure, ventricular arrhythmias, and sudden cardiac death (SCD) when compared to Caucasians.¹⁻³ While implantable cardioverter-defibrillators (ICDs) effectively reduce mortality from SCD in heart failure patients,^{4,5} the ability to identify prospectively patients who will derive benefit from these therapies is lacking. Current guidelines, based upon clinical trials, fail to identify the majority of patients who will suffer SCD.⁶ New risk stratification algorithms are urgently needed, especially for African Americans who have been underrepresented in clinical trials. Genetic based risk stratification offers the potential for both identifying high-risk patients who do not qualify for primary prevention strategies and excluding low-risk individuals who do.

The present invention overcomes previous shortcomings in the art by identifying significant statistical associations between genetic markers and sudden cardiac death. Thus, the present invention provides methods and compositions for identifying a subject at increased risk of sudden cardiac death by detecting the genetic markers of this invention in the subject.

### SUMMARY OF THE INVENTION

In one aspect, the present invention provides a method of identifying a subject as having an increased risk of sudden cardiac death, comprising detecting in a nucleic acid sample of the subject an A allele at single nucleotide polymorphism rs7626962 or a T allele at single polymorphism rs7629265, wherein detection of either of said alleles identifies the subject as having an increased risk of sudden cardiac death.

In a further aspect, the present invention provides a method of identifying a subject as having an increased risk of cardiac arrhythmia (e.g., a subject with reduced left ventricular function), comprising detecting in a nucleic acid sample of the subject an A allele at single nucleotide polymorphism rs7626962 or a T allele at single polymorphism rs7629265, wherein detection of said allele identifies the subject as having an increased risk of cardiac arrhythmia.

As an additional aspect, the present invention provides a method of identifying a subject for whom implantable cardioverter defibrillator (ICD) therapy would be beneficial, comprising detecting in a nucleic acid sample of the subject an A allele at single nucleotide polymorphism rs7626962 or a T allele at single polymorphism rs7629265, wherein detection of said allele identifies the subject for whom ICD therapy would be beneficial.

In yet further aspects, the present invention provides a kit comprising oligonucleotides to detect the A allele of single nucleotide polymorphism rs7626962 in a nucleic acid sample and/or the T allele of single nucleotide polymorphism rs7629265

### BRIEF DESCRIPTION OF THE DRAWINGS

FIG. 1: Prevalence of carriers for the minor alleles indicated, stratified by ICD therapy.

FIG. 2: Kaplan-Meier curve for appropriate ICD therapy stratified by genotype (S1103Y carriers).

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery of particular alleles of single nucleotide polymorphisms (SNPs) that are statistically associated with an increased risk of sudden cardiac death and cardiac arrhythmia in subjects (e.g., black human subjects) with impaired left ventricle systolic function. There are numerous benefits of carrying out the methods of this invention to identify a subject (e.g., a subject with reduced left ventricular function) as having an increased risk of sudden cardiac death, including but not limited to, identifying subjects who are good candidates for ICD therapy, particularly among subjects who do not meet the criteria for ICD therapy under current parameters and avoiding such procedures in subjects least likely to benefit from them.

Thus, in one aspect, the present invention provides a method of identifying a subject (e.g., a subject with reduced left ventricular function) as having an increased risk of sudden cardiac death, comprising detecting in a nucleic acid sample of the subject an A allele at single nucleotide polymorphism rs7626962, wherein the detection of said allele identifies the subject as having an increased risk of sudden cardiac death.

The present invention further provides a method of identifying a subject as having an increased risk of sudden cardiac death, comprising detecting in a nucleic acid sample of the subject an allele in linkage disequilibrium (LD) with the A allele at single nucleotide polymorphism rs7626962. Alleles in LD with the A allele at single nucleotide polymorphism rs7626962 include a T allele at single nucleotide polymorphism rs7629265. Such alleles can be detected individually as well as in combination. Thus, in some embodiments, when analyzed in combination, the combination can comprise detection of the A allele at rs7626962 in addition to detection of the T allele at rs7629265.

The presence of an A allele at single nucleotide polymorphism rs7626962 in the nucleotide sequence of the SCN5A gene results in a serine to tyrosine substitution at amino acid 1103 (S1103Y) in the amino acid sequence of the Naᵥ1.5 protein that has the amino acid sequence of GenBank® Database Accession No. NP_{—}932173:

| | |
|---|---|
| 1 | manfllprgt ssfrrftres laaiekrmae kqargsttlq esreglpeee aprpqldiqa |
| 61 | skklpdlygn ppqeligepl edldpfystq ktfivinkgk tifrfsatna lyvlspfhpi |
| 121 | rraavkilvh slfnmlimct iltncvfmaq hdpppwtkyv eytftaiytf eslvkilarg |
| 181 | fclhaftflr dpwnwldfsv iimayttefv dlgnvsalrt frvlralkti svisglktiv |
| 241 | galiqsvkkl advmvltvfc lsvfaliglq lfmgnlrhkc vrnftalngt ngsveadglv |
| 301 | wesldlylsd penyllkngt sdvllcgnss dagtcpegyr clkagenpdh gytsfdsfaw |
| 361 | aflalfrlmt qdcwerlyqq tlrsagkiym iffmlviflg sfylvnlila vvamayeeqn |
| 421 | qatiaeteek skrfqeamem lkkehealti rgvdtvsrss lemsplapvn sherrskrrk |
| 481 | rmssgteecg edrlpksdse dgpramnhls ltrglsrtsm kprssrgsif tfrrrdlgse |
| 541 | adfaddenst ageseshhts llvpwplrrt saqgqpspgt sapghalhgk knstvdcngv |
| 601 | vsllgagdpe atspgshllr pvmlehppdt ttpseepggp gmltsqapcv dgfeepgarq |
| 661 | ralsavsvlt saleeleesr hkcppcwnrl agryliwecc plwmsikqgv klvvmdpftd |
| 721 | ltitmcivln tlfmalehyn mtsefeemlq vgnlvftgif taemtfkiia ldpyyyfqqg |
| 781 | wnifdsiivi lslmelglsr msnlsvlrsf rllrvfklak swptlntlik iignsvgalg |
| 841 | nltlvlaiiv fifavvgmql fgknyselrd sdsgllprwh mmdffhafli ifrilcgewi |
| 901 | etmwdcmevs gqslcllvfl lvmvignlvv lnlflallls sfsadnltap dedremnnlq |
| 961 | lalariqrgl rfvkrttwdf ccgllrqrpq kpaalaaqgq lpsciatpys ppppetekvp |
| 1021 | ptrketrfee geqpgqgtpg dpepvcvpia vaesdtddqe edeenslgte eesskqqesq |
| 1081 | pvsggpeapp dsrtwsqvsa tasseaeasa sqadwrqqwk aepqapgcge tpedscsegs |
| 1141 | tadmtntael leqipdlgqd vkdpedcfte gcvrrcpcca vdttqapgkv wwrlrktcyh |
| 1201 | ivehswfetf iifmillssg alafediyle erktikvlle yadkmftyvf vlemllkwva |
| 1261 | ygfkkyftna wcwldflivd vslvslvant lgfaemgpik slrtlralrp lralsrfegm |
| 1321 | rvvvnalvga ipsimnvllv clifwlifsi mgvnlfagkf grcinqtegd lplnytivnn |
| 1381 | ksqceslnlt gelywtkvkv nfdnvgagyl allqvatfkg wmdimyaavd srgyeeqpqw |
| 1441 | eynlymyiyf vifiifgsff tlnlfigvii dnfnqqkkkl ggqdifmtee qkkyynamkk |
| 1501 | lgskkpqkpi prplnkyqgf ifdivtkqaf dvtimflicl nmvtmmvetd dqspekinil |
| 1561 | akinllfvai ftgecivkla alrhyyftns wnifdfvvvi lsivgtvlsd iiqkyffspt |
| 1621 | lfrvirlari grilrlirga kgirtllfal mmslpalfni glllflvmfi ysifgmanfa |
| 1681 | yvkweagidd mfnfqtfans mlclfqitts agwdgllspi lntgppycdp tlpnsngsrg |
| 1741 | dcgspavgil ffttyiiisf livvnmyiai ilenfsvate esteplsedd fdmfyeiwek |
| 1801 | fdpeatqfie ysvlsdfada lseplriakp ngislinmdl pmvsgdrihc mdilfaftkr |
| 1861 | vlgesgemda lkiqmeekfm aanpskisye pitttlrrkh eevsamviqr afrrhllqrs |
| 1921 | lkhasflfrq qagsglseed aperegliay vmsenfsrpl gppssssiss tsfppsydsv |
| 1981 | tratsdnlqv rgsdyshsed ladfppspdr dresiv, |

The presence of a T allele at single nucleotide polymorphism rs7629265 in the nucleotide sequence of the SCN5A gene results in a serine to phenylalanine substitution at amino acid 1103 (S1103F) in the amino acid sequence of the Naᵥ1.5 protein of GenBank® Database Accession No. NP_{—}932173 as shown above.

In some embodiments of this invention, the subject can be homozygous for the A allele at single nucleotide polymorphism rs7626962. In other embodiments, the subject can be heterozygous for the A allele at single nucleotide polymorphism rs7626962. The presence of the A allele, either homozygously or heterozygously, at single nucleotide polymorphism rs7626962 identifies the subject as having an increased risk of sudden cardiac death and/or an increased risk of cardiac arrhythmia.

Furthermore, in some embodiments of this invention, the subject can be homozygous for the T allele at single nucleotide polymorphism rs7629265. In other embodiments, the subject can be heterozygous for the T allele at single nucleotide polymorphism rs7629265. The presence of the T allele, either homozygously or heterozygously, at single nucleotide polymorphism rs7629265 identifies the subject as having an increased risk of sudden cardiac death and/or an increased risk of cardiac arrhythmia. In the methods provided herein wherein a combination of alleles is analyzed, the subject can be heterozygous or homozygous for any given allele in any combination relative to the other allele(s) in the combination.

It is further contemplated that the methods of this invention can be carried out to identify a subject for whom implantable cardioverter defibrillator (ICD) therapy would be beneficial, by detecting the A allele of SNP rs7626962 and/or detecting the T allele of SNP rs7629265 in nucleic acid from the subject. The presence of the A allele, either homozygously or heterozygously, at single nucleotide polymorphism rs7626962 identifies the subject as a subject for whom ICD therapy would be beneficial. The presence of the T allele, either homozygously or heterozygously, at single nucleotide polymorphism rs7629265 identifies the subject as a subject for whom ICD therapy would be beneficial.

In some embodiments, the subject of this invention can be a subject with impaired left ventricular function. In some embodiments, the subject can be a subject with mildly reduced left ventricular function, 35%<EF<55%. Current guidelines for ICD implantation for secondary prevention of sudden death in patients with heart failure/reduced left ventricular function are for patients with an ejection fraction (EF; the fraction of blood in the heart expelled during each heart beat) of <35%. In the present invention it is contemplated that patients with less severe reduction in pump ability (i.e., EF of >35%, such as for example, 35%<EF<55%) can be identified as subjects for whom ICD therapy would be beneficial based on the presence of an allele of a SNP of this invention in nucleotide acid of the subject. These subjects are currently not considered for ICD therapy, which is based on a strict cutoff of EF<35%.

In further aspects, the present invention provides a kit for carrying out the methods of this invention, wherein the kit can comprise oligonucleotides (e.g., primers, probes, primer/probe sets, etc.), reagents, buffers, etc., as would be known in the art, for the detection of the polymorphisms and/or alleles of this invention in a nucleic acid sample. For example, a primer or probe can comprise a contiguous nucleotide sequence that is complementary (e.g., fully (100%) complementary or partially (50%, 60%, 70%, 80%, 90%, 95%, etc.) complementary) to a region comprising an allele of this invention. In particular embodiments, a kit of this invention will comprise primers and probes that allow for the specific detection of the alleles of this invention. Such a kit can further comprise blocking probes, labeling reagents, blocking agents, restriction enzymes, antibodies, sampling devices, positive and negative controls, etc., as would be well known to those of ordinary skill in the art. Thus, in some embodiments, the present invention provides a kit comprising oligonucleotides to detect the A allele of single nucleotide polymorphism rs7626962 in and/or the T allele of single nucleotide polymorphism rs7629265 in a nucleic acid sample.

### DEFINITIONS

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “sudden cardiac death” describes death from coronary heart disease deaths in persons dying within 1 hour of onset of cardiovascular symptoms (event witnessed), or within 24 h of having been observed alive and symptom free (unwitnessed) or survivors of cardiac arrest.

Also as used herein the term “cardiac arrhythmia” means an irregular and life-threatening cardiac ventricular rhythm, such as ventricular tachycardia or ventricular fibrillation.

In addition, as used herein the terms “impaired left ventricle function,” and “reduced left ventricle systolic function” describe a state in which the left ventricle (the main pumping chamber of the heart) is weakened, and thereby compromised in its ability to expel blood to the body.

Furthermore, as used herein, the term “heart failure” means a condition in which a subject has symptoms of impaired left ventricular function, such as shortness of breath (dyspnea) during exertion or when lying down, fatigue and weakness, swelling (edema) in legs, ankles and feet, rapid or irregular heartbeat, reduced ability to exercise, persistent cough or wheezing with white or pink blood-tinged phlegm, swelling of your abdomen (ascites), sudden weight gain from fluid retention, lack of appetite and nausea, difficulty concentrating or decreased alertness.

As used herein, “implantable cardioverter defibrillator (ICD) therapy” describes a device that detects a life-threatening cardiac arrhythmia and delivers therapy (antitachycardia pacing and/or defibrillation in an attempt to reverse the cardiac arrhythmia.

A subject of this invention can be any subject that is susceptible to sudden cardiac death and/or cardiac arrhythmia and in particular embodiments, the subject of this invention is a human subject. The subject of this invention can be a black human subject. By “black” is meant that the subject is of African descent, including a subject of sub-Saharan African descent, which includes but is not limited to African American, African, African European, African Australian, African Asian, African Caribbean, etc., as would be known in the art.

Also as used herein, “linked” describes a region of a chromosome that is shared more frequently in family members or members of a population manifesting a particular phenotype and/or affected by a particular disease or disorder, than would be expected or observed by chance, thereby indicating that the gene or genes or other identified marker(s) within the linked chromosome region contain or are associated with an allele that is correlated with the phenotype and/or presence of a disease or disorder, or with an increased or decreased likelihood of the phenotype and/or of the disease or disorder. Once linkage is established, association studies (linkage disequilibrium) can be used to narrow the region of interest or to identify the marker (e.g., allele or haplotype) correlated with the phenotype and/or disease or disorder.

Furthermore, as used herein, the term “linkage disequilibrium” or “LD” refers to the occurrence in a population of two or more (e.g., 3, 4, 5, 6, 7, 8, 9, 10, etc.) linked alleles at a frequency higher or lower than expected on the basis of the gene frequencies of the individual genes. Thus, linkage disequilibrium describes a situation where alleles occur together more often than can be accounted for by chance, which indicates that the two or more alleles are physically close on a DNA strand.

The term “genetic marker” or “polymorphism” as used herein refers to a characteristic of a nucleotide sequence (e.g., in a chromosome) that is identifiable due to its variability among different subjects (i.e., the genetic marker or polymorphism can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein, etc., as would be well known to one of ordinary skill in the art).

A “single nucleotide polymorphism” (SNP) in a nucleotide sequence is a genetic marker that is polymorphic for two (or in some case three or four) alleles. SNPs can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

In some embodiments, the term genetic marker is also intended to describe a phenotypic effect of an allele or haplotype, including for example, an increased or decreased amount of a messenger RNA, an increased or decreased amount of protein, an increase or decrease in the copy number of a gene, production of a defective protein, tissue or organ, etc., as would be well known to one of ordinary skill in the art.

An “allele” as used herein refers to one of two or more alternative forms of a nucleotide sequence at a given position (locus) on a chromosome. An allele can be a nucleotide present in a nucleotide sequence that makes up the coding sequence of a gene and/or an allele can be a nucleotide in a non-coding region of a gene (e.g., in a genomic sequence). A subject's genotype for a given gene is the set of alleles the subject happens to possess. As noted herein, an individual can be heterozygous or homozygous for any allele of this invention.

Also as used herein, a “haplotype” is a set of alleles on a single chromatid that are statistically associated. It is thought that these associations, and the identification of a few alleles of a haplotype block, can unambiguously identify all other alleles in its region. The term “haplotype” is also commonly used to describe the genetic constitution of individuals with respect to one member of a pair of allelic genes; sets of single alleles or closely linked genes that tend to be inherited together.

The terms “increased risk” and “decreased risk” as used herein define the level of risk that a subject has of sudden cardiac death and/or cardiac arrhythmia, as compared to a control subject that does not have the polymorphisms and alleles of this invention in the control subject's nucleic acid.

A sample of this invention can be any sample containing nucleic acid of a subject, as would be well known to one of ordinary skill in the art. Nonlimiting examples of a sample of this invention include a cell, a body fluid, a tissue, a washing, a swabbing, etc., as would be well known in the art.

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. In some embodiments, the nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence or nucleic acid molecule that is not immediately contiguous with nucleotide sequences or nucleic acid molecules with which it is immediately contiguous (one on the 5′ end and one on the 3′ end) in the naturally occurring genome of the organism from which it is derived or in which it is detected or identified. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid encoding an additional polypeptide, peptide sequence and/or other gene product.

The term “isolated” can also refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about five nucleotides to about 500 nucleotides (e.g. 5, 6, 7, 8, 9, 10, 12, 15, 18, 20, 21, 22, 25, 30, 35, 40, 45, 50, 55, 60, 65, 70, 75, 80, 85, 90, 100, 125, 150, 175, 200, 250, 300, 350, 400, 450 or 500 nucleotides). In some embodiments, for example, an oligonucleotide can be from about 15 nucleotides to about 30 nucleotides, or about 20 nucleotides to about 25 nucleotides, which can be used, for example, as a primer in a polymerase chain reaction (PCR) amplification assay and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as primers and/or probes. Such fragments or oligonucleotides can be detectably labeled or modified, for example, to include and/or incorporate a restriction enzyme cleavage site when employed as a primer in an amplification (e.g., PCR) assay.

The detection of a polymorphism, genetic marker or allele of this invention can be carried out according to various protocols standard in the art and as described herein for analyzing nucleic acid samples and nucleotide sequences, as well as identifying specific nucleotides in a nucleotide sequence.

For example, nucleic acid can be obtained from any suitable sample from the subject that will contain nucleic acid and the nucleic acid can then be prepared and analyzed according to well-established protocols for the presence of genetic markers according to the methods of this invention.

In some embodiments, analysis of the nucleic acid can be carried by amplification of the region of interest, according to protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

In some embodiments of this invention, detection of an allele or combination of alleles of this invention can be carried out by an amplification reaction and single base extension. In particular embodiments, the product of the amplification reaction and single base extension is spotted on a silicone chip.

In yet additional embodiments, detection of an allele or combination of alleles of this invention can be carried out by matrix-assisted laser desorption/ionization-time of flight mass spectrometry (MALDI-TOF-MS).

It is further contemplated that the detection of an allele or combination of alleles of this invention can be carried out by various methods that are well known in the art, including, but not limited to nucleic acid sequencing, hybridization assay, restriction endonuclease digestion analysis, electrophoresis, and any combination thereof.

The genetic markers (e.g., alleles) of this invention are correlated with (i.e., identified to be statistically associated with) sudden cardiac death and/or cardiac arrhythmia as described herein according to methods well known in the art and as disclosed in the Examples provided herein for statistically correlating genetic markers with various phenotypic traits, including disease states and pathological conditions as well as determining levels of risk associated with developing a particular phenotype, such as a disease or pathological condition. In general, identifying such correlation involves conducting analyses that establish a statistically significant association and/or a statistically significant correlation between the presence of a genetic marker or a combination of markers and the phenotypic trait in a population of subjects and controls (e.g., a population of subjects in whom the phenotype is not present or has not been detected). The correlation can involve one or more than one genetic marker of this invention (e.g., two, three, four, five, or more) in any combination. An analysis that identifies a statistical association (e.g., a significant association) between the marker or combination of markers and the phenotype establishes a correlation between the presence of the marker or combination of markers in a population of subjects and the particular phenotype being analyzed. A level of risk (e.g., increased or decreased) can then be determined for an individual on the basis of such population-based analyses.

In some embodiments, the methods of correlating genetic markers with disease states and effective treatments and/or therapies of this invention can be carried out using a computer database. Thus the present invention provides a computer-assisted method of identifying a proposed treatment and/or appropriate treatment for a subject carrying a genetic marker of this invention. The method involves the steps of (a) storing a database of biological data for a plurality of subjects, the biological data that is being stored including for each of said plurality of subjects, for example, (i) a treatment type, (ii) at least one genetic marker associated with sudden cardiac death or cardiac arrhythmia and (iii) at least one disease progression measure for sudden cardiac death or cardiac arrhythmia from which treatment efficacy can be determined; and then (b) querying the database to determine the correlation between the presence of said genetic marker and the effectiveness of a treatment type, to thereby identify a proposed treatment as an effective treatment.

In some embodiments, treatment information for a subject is entered into the database (through any suitable means such as a window or text interface), genetic marker information for that subject is entered into the database, and disease progression information is entered into the database. These steps are then repeated until the desired number of subjects has been entered into the database. The database can then be queried to determine whether a particular treatment is effective for subjects carrying a particular marker or combination of markers, not effective for subjects carrying a particular marker or combination of markers, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### Examples

Abstract.

Risk stratifying heart failure patients for primary prevention implantable cardioverter-defibrillators (ICDs) remains a challenge, especially for African Americans (i.e., blacks), who have an increased incidence of sudden cardiac death but have been underrepresented in clinical trials. These studies were carried out to determine whether the S1103Y cardiac sodium channel SCN5A variant influences the propensity for ventricular arrhythmias in African American patients with heart failure and reduced ejection fraction.

112 African Americans with ejection fractions (EF)<35% receiving primary prevention ICDs were identified from the Duke Electrophysiology Genetic and Genomic Studies (EPGEN) biorepository and followed for appropriate ICD therapy (either antitachycardia pacing or shock) for documented sustained ventricular tachycardia or fibrillation. The S1103Y variant was over-represented in patients receiving appropriate ICD therapy compared to subjects who did not (35% vs. 13%, p=0.02). Controlling for baseline characteristics, the adjusted hazard ratio using a Cox Proportional Hazard Model for ICD therapy in Y1103 allele carriers was 4.33 (95% CI 1.60-11.73, p=<0.01). There was no difference in mortality between carriers and non-carriers.

This is the first report that the S1103Y variant is associated with a higher incidence of ventricular arrhythmias in African Americans with heart failure and reduced ejection fraction.

Study Population.

The Duke Electrophysiology Genetic and Genomic Studies (EPGEN) biorepository is a prospective single-center repository that archives DNA, RNA, and protein samples obtained at the time of an electrophysiological evaluation or intervention.⁷ Demographic, laboratory and cardiovascular risk factor data are collected at the time of ICD implantation. Device type was determined by the implanting operator and was not restricted by manufacturer. ICD programming and follow-up intervals were left to the discretion of the treating electrophysiologist. Between May 2005 and April 2008, 137 consecutive self-identified African American patients aged 18 or older undergoing ICD placement for purposes of primary prevention with a left ventricular ejection fraction (EF) <35%, were identified. Twenty-three subjects were excluded because of insufficient clinical follow-up variables or insufficient DNA. Two were excluded because of suspected or confirmed inherited arrhythmia syndromes.

Follow-Up and Classification of Events.

Patients were classified as having received appropriate ICD therapy if they experienced antitachycardia pacing or shock for documented sustained ventricular tachycardia or ventricular fibrillation. Patients that only received inappropriate ICD therapy or did not receive any antitachycardia pacing or shock were classified as having no appropriate ICD therapy. Events were adjudicated at the time of device interrogation by review of the treating electrophysiologist, who was blinded to the genotype. The research protocol was approved by the Duke University Institutional Review Board and all patients consented to use of their samples for the EPGEN biorepository.

Statistical Analysis.

Standard descriptive statistics were used, including percentages for discrete variables and means for continuous variables. Baseline characteristics were tested for significance with the Chi-square statistic for categorical variables and with the Student's t-test for continuous variables. Hardy-Weinberg Equilibrium for the two single nucleotide polymorphisms (SNPs) was tested using Chi-square. Allele frequencies were compared between patients receiving appropriate ICD therapy and patients that did not receive appropriate ICD therapy using Chi-square or Fisher's Exact Test where appropriate. The primary analysis was a time-to-event analysis to assess the relationship between time to appropriate ICD therapy and genotype, and thus a life-table Kaplan-Meier survival analysis was performed using the log-rank statistic. Risk relationships were characterized as hazard ratios and 95% confidence intervals, generated with the use of a multivariable Cox Proportional Hazard model with covariates known to be associated with mortality and sudden cardiac death: age, gender, history of coronary artery disease (CAD), hyperlipidemia, hypertension, history of tobacco use, diabetes, and genotype. Two sided P-values of <0.05 were considered as statistically significant. All statistical analyses were performed with SAS version 9.2 (SAS Institute, Inc., Cary, N.C.).

Genotyping.

DNA was extracted using a PureGene kit (Gentra Systems, Minneapolis, Minn.) following manufacturer's standard protocol. Genotypes for GenBank® Database Accession No. rs7626962 (S1103Y) were determined using the 7900HT Taqman® genotyping system (Applied Biosystems, Foster City, Calif.), which incorporates a standard PCR-based, dual fluorophore, allelic discrimination assay. Assays were purchased from Applied Biosystems. QC samples, composed of 12 reference controls, were included in each quadrant of the plate. SNPs showing mismatches on QC samples were reviewed by an independent supervisor. Both SNPs were successfully genotyped for all of the individuals in the study. Error rate estimates for SNPs meeting QC benchmarks were <0.2%.

Patient Characteristics.

A total of 112 African Americans who received ICDs for primary prevention were included. Mean follow-up was 865 days (interquartile range 585-1131 days). The mean age was 62.8 and 34% of patients were women. Twenty-three patients (21%) received appropriate ICD therapy and 89 patients (79%) did not receive appropriate ICD therapy. Twelve of the 23 patients (52%) that received appropriate ICD therapy experienced ATP only as their classifying event. Baseline characteristics with respect to diabetes, tobacco use, hypertension, hyperlipidemia, atrial fibrillation, serum potassium level, NYHA class, ejection fraction, maximum LV wall thickness, or medications at time of enrollment did not differ significantly between patients that received appropriate ICD therapy and those that did not (Table 1). The corrected QT duration did not differ between the two groups (463 vs. 465 ms, p=0.41). There was a higher percentage of patients classified as having non-ischemic cardiomyopathy among those receiving appropriate ICD therapy (74% vs. 48%, p=0.02).

Association of S1103Y and ICD Therapy.

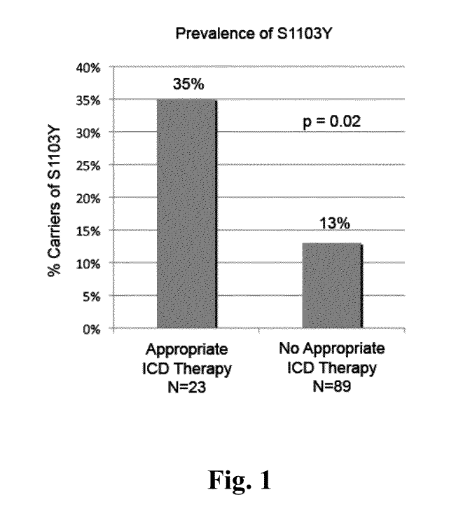

Genotyping revealed a Y1103 allele frequency of 0.09 with 92 patients homozygous for the S1103 allele (SS), 19 heterozygous (SY) and 1 homozygous for the Y1103 allele (YY) (Table 2). There was no departure from Hardy-Weinberg equilibrium. Chi-Square analyses using a dominant model (at least one copy of the Y1103 allele) revealed a significant over representation of the Y1103 allele in patients that received appropriate ICD therapy versus no appropriate ICD therapy, (8 of 23 [35%] vs. 12 of 89 [13%], p=0.02) (FIG. 1). The presence of the Y1103 allele correlated to an unadjusted odds ratio for appropriate ICD therapy of 3.42 (95% CI 1.20-9.80, p=0.02). Although study-independent, genotype-blinded electrophysiologists adjudicated all ICD events, a sensitivity analysis excluding the 24 subjects that received only inappropriate ICD therapy was performed to account for possible errors in adjudication. Consistent with the primary analysis, this revealed an unadjusted odds ratio of 3.32 (95% CI 1.09-10.07, p=0.03) for appropriate ICD therapy in Y1103 carriers. When analyzed by allele status, there was no significant difference in baseline characteristics (Table 3).

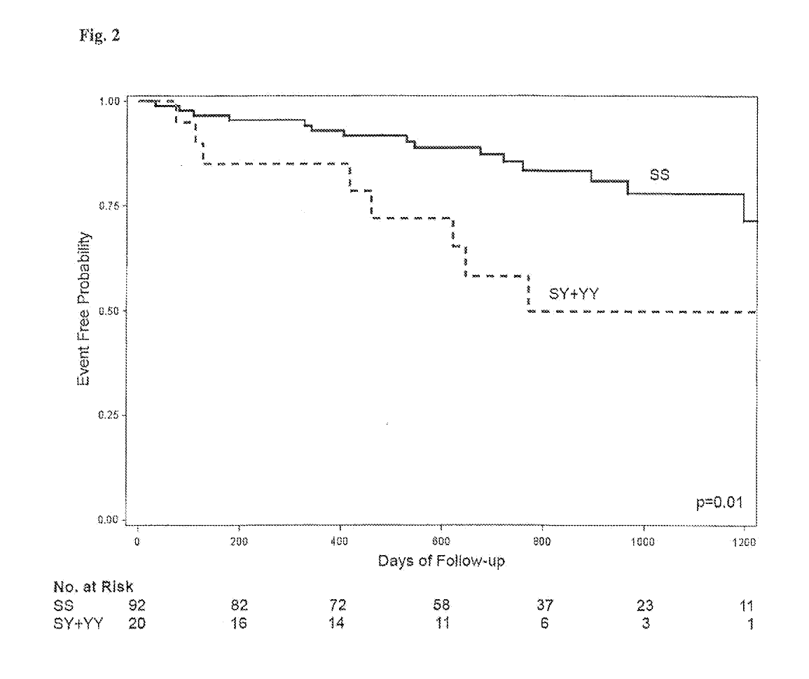

To assess the relationship between genotype and time to appropriate ICD therapy, a time-to-event survival analysis was performed. Kaplan-Meier curves diverged early and continued to diverge throughout the follow-up period showing a higher incidence of appropriate ICD therapy in Y1103 carriers (FIG. 2, log-rank p=0.01). The mean time to appropriate ICD therapy for carriers was 609±59 days compared to 1057±36 days for non-carriers. Utilizing a Cox Proportional Hazard Model, carriers of the Y1103 allele had an age and gender adjusted hazard ratio of 3.50 (95% CI 1.38-8.85, p=<0.01) for ICD therapy compared to non-carriers (Table 4). An additional multivariable Cox Proportional Hazard Model was performed adjusting for age and gender as well as history of CAD, hyperlipidemia, hypertension, history of tobacco use, and diabetes and resulted in an increased hazard ratio of 4.33 (95% CI 1.60-11.73, p=<0.01) for appropriate ICD therapy in carriers of the Y1103 allele (Table 4).

There were four deaths among patients that received appropriate ICD therapy (one carrier of the Y1103 allele) and 15 deaths in those patients that did not receive appropriate ICD therapy (three were carriers of the Y1103 allele). There was no association between all-cause mortality and Y1103 carrier status, p=0.69.

This is believed to be the first report that the S1103Y variant in SCN5A is associated with a higher incidence of ventricular arrhythmias in African Americans with heart failure and reduced ejection fraction. The specific association between the S1103Y genotype and an increased risk of arrhythmia in heart failure may have particular significance as a novel risk factor for African Americans. In combination with traditional risk stratification tools such as EF, ECG analysis, and medical history,⁸ the addition of a genetic component could further strengthen the ability to identify heart failure patients at highest risk for sudden cardiac death and to identify mechanisms to prevent it. Given the relatively high prevalence of the S1103Y variant, this finding has a potentially large impact for the African American community.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

### REFERENCES

1. Heiat et al. Representation of the elderly, women, and minorities in heart failure clinical trials. Arch Intern Med. 2002; 162:1682-1688
2. Leyris et al. RGK GTPase-dependent CaV2.1 Ca2+ channel inhibition is independent of CaV {beta}-subunit-induced current potentiation. FASEB J. 2009; 23:2627-2638
3. Lee et al. Ca2+/calmodulin-dependent facilitation and inactivation of P/Q-type Ca2+ channels. J. Neurosci. 2000; 20:6830-6838
4. Catterall et al. Inherited neuronal ion channelopathies: New windows on complex neurological diseases. J. Neurosci. 2008; 28:11768-11777
5. Bardy et al. Amiodarone or an implantable cardioverter-defibrillator for congestive heart failure. N Engl J Med. 2005; 352:225-237
6. Epstein et al. ACC/AHA/HRS 2008 guidelines for device-based therapy of cardiac rhythm abnormalities: A report of the American College of Cardiology/American Heart Association task force on practice guidelines (writing committee to revise the ACC/AHA/NASPE 2002 guideline update for implantation of cardiac pacemakers and antiarrhythmia devices) developed in collaboration with the American Association for Thoracic Surgery and Society of Thoracic Surgeons. J Am Coll Cardiol. 2008; 51:e1-62
7. Koontz et al. Rationale and design of the Duke electrophysiology genetic and genomic studies (EPGEN) biorepository. Am Heart J. 2009; 158:719-725
8. Atwater et al. Usefulness of the Duke sudden cardiac death risk score for predicting sudden cardiac death in patients with angiographic (>75% narrowing) coronary artery disease. Am J Cardiol. 2009; 104:1624-1630

TABLE 1
Baseline Characteristics Stratified by ICD Therapy

| | | | No |
|---|---|---|---|
| | Entire | Appropriate | Appropriate |
| | Cohort | ICD Therapy | ICD Therapy |
| Characteristic* | (n = 112) | (n = 23) | (n = 89) |
| Age, Mean (SD), y | 63 (12) | 64 (9) | 63 (13) |
| Male, No. (%) | 74 (66) | 18 (78) | 56 (63) |
| Medical History | | | |
| History of CAD, No. (%)+ | 52 (46) | 6 (26) | 46 (52) |
| History of Diabetes, No. (%) | 57 (51) | 11 (46) | 46 (52) |
| Tobacco Use, No. (%) | 59 (53) | 12 (52) | 47 (53) |
| History of Hypertension, | 101 (90) | 22 (96) | 79 (89) |
| No. (%) | | | |
| History of Hyperlipidemia, | 81 (72) | 18 (78) | 63 (71) |
| No. (%) | | | |
| History of Atrial Fibrillation, | 51 (46) | 9 (39) | 42 (47) |
| No. (%) | | | |
| NYHA Class, Mean (SD) | 2.4 (0.6) | 2.3 (0.7) | 2.4 (0.6) |
| Echo Parameters | | | |
| Ejection Fraction, Mean (SD), % | 25 (6) | 24 (7) | 25 (6) |
| Maximal LV Wall Thickness, | 1.4 (0.3) | 1.4 (0.3) | 1.4 (0.3) |
| Mean (SD), cm | | | |
| ECG Parameters | | | |
| QTc, Mean (SD), ms | 464 (41) | 463 (45) | 465 (40) |
| Lab Values | | | |
| Serum Potassium at Enrollment, | 4.2 (0.5) | 4.1 (0.4) | 4.2 (0.5) |
| Mean (SD), mmol/L | | | |
| Medication | | | |
| Beta Blockers, No. (%) | 104 (93) | 22 (96) | 82 (92) |
| ACE Inhibitors, No. (%) | 79 (70) | 19 (82) | 60 (67) |
| Diuretics, No. (%) | 91 (81) | 19 (83) | 72 (81) |
| Aspirin, No. (%) | 98 (88) | 19 (83) | 79 (89) |
| Digoxin, No. (%) | 28 (25) | 6 (26) | 22 (25) |

QTc, QT Interval corrected using Bazett's Formula (QTc = QT/√RR).
*P > 0.05 for all variables except history of CAD
+P = 0.03

TABLE 2
Genotype Frequency of the S1103Y variant
by ICD therapy

| | Genotype Frequency | | |
|---|---|---|---|
| | rs7626962-S1103Y | | |
| | CC | CA | AA |
| Appropriate ICD therapy | 15 | 8 | 0 |
| No Appropriate ICD therapy | 77 | 11 | 1 |

TABLE 3
Clinical Characteristics and Events Stratified by Y1103 Allele Status

| | Y1103 | Y1103 |
|---|---|---|
| | Carrier | Non Carrier |
| Characteristic | (n = 20) | (n = 92) |
| Appropriate ICD Events, No. (%)* | 8 (40) | 15 (16) |
| History of CAD, No. (%) | 9 (45) | 43 (47) |
| Ejection Fraction, Mean (SD) | 26 (7) | 24 (6) |
| QTc, Mean (SD), ms | 458 (47) | 466 (40) |

*p = 0.02

TABLE 4
Multivariable Cox Proportional Hazard Model

| | HR (95% CI) for ICD | |
|---|---|---|
| Variant | Therapy | P-value |
| S1103Y | | |
| Age and gender adjusted | 3.50 (1.38-8.85) | <0.01 |
| Multivariable model | 4.33 (1.60-11.73) | <0.01 |

Multivariable model controlled for age, gender, history of CAD, HTN, HL, TOB, and Diabetes.

That which is claimed is:

1. A method of identifying a subject as having an increased risk of sudden cardiac death and/or an increased risk of cardiac arrhythmia, comprising detecting in a nucleic acid sample of the subject an A allele at single nucleotide polymorphism rs7626962 and/or a T allele at single polymorphism rs7629265, wherein detection of said allele(s) identifies the subject as having an increased risk of sudden cardiac death and/or an increased risk of cardiac arrhythmia.

2. A method of identifying a subject for whom implantable cardioverter defibrillator (ICD) therapy would be beneficial, comprising detecting in a nucleic acid sample of the subject an A allele at single nucleotide polymorphism rs7626962 and/or a T allele at single polymorphism rs7629265, wherein detection of said allele(s) identifies the subject as a subject for whom ICD therapy would be beneficial.

3. The method of claim 1, wherein the subject is a black human subject.

4. The method of claim 1, wherein the subject has impaired left ventricular function.

5. The method of claim 4, wherein the subject has mildly reduced left ventricular function, 35%<EF<55%.

6. The method of claim 1, wherein detecting is carried out by an amplification reaction.

7. The method of claim 1, wherein detecting is carried out by an amplification reaction and single base extension.

8. The method of claim 7, wherein the product of the amplification reaction and single base extension is spotted on a silicone chip.

9. The method of claim 1, wherein detecting is carried out by matrix-assisted laser desorption/ionization-time of flight mass spectrometry (MALDI-TOF-MS).

10. The method of claim 6, wherein the amplification reaction is a polymerase chain reaction.

11. The method of any claim 1, wherein detecting is carried out by sequencing, hybridization, restriction endonuclease digestion analysis, electrophoresis, or any combination thereof.

12. A kit comprising oligonucleotides to detect an A allele at single nucleotide polymorphism rs7626962 and/or a T allele at single polymorphism rs7629265 in a nucleic acid sample.


SEQUENCE LISTING (1 sequence)

SEQ ID NO: 1 - protein, 2016 aa, Homo sapiens [sequence omitted]